(19) United States
(12) Patent Application Publication
(10) Pub. No.: US 2014/0162941 A1
(43) Pub. Date: Jun. 12, 2014

(54) PROCESS FOR PREPARING BIOLOGICAL SAMPLES

(71) Applicants: Leonard Weisman, Houston, TX (US); Lingkun Kong, Pearland, TX (US)

(72) Inventors: Leonard Weisman, Houston, TX (US); Lingkun Kong, Pearland, TX (US)

(73) Assignee: BAYLOR COLLEGE OF MEDICINE, Houston, TX (US)

(21) Appl. No.: 14/235,350

(22) PCT Filed: Jul. 26, 2012
(86) PCT No.: PCT/US12/48292

§ 371 Date: Feb. 10, 2014

Related U.S. Application Data
(60) Provisional application No. 61/512,141, filed on Jul. 27, 2011.

(51) Int. Cl. C12Q 1/68

(52) U.S. Cl. CPC C12Q 1/689
USPC 514/3.1; 435/6.12; 435/6.15; 435/6.11; 506/9

(57) ABSTRACT

Methods and compositions for preparation of a sample, including a sample to be analyzed for the presence of one or more pathogens. Transport of a non-diluted sample from an individual suspected of having the pathogen and transfer of the sample directly into a nucleic acid extraction buffer occurs in a single step. The process is an improvement over known methods because it provides accurate, rapid analysis that utilizes fewer steps and/or reagents from methods used in the art. In specific embodiments, it has one or more of the following characteristics: 1) it is a one-step process; 2) it eliminates dilution of the sample; 3) smaller sample sizes are employed; 4) fewer reagents are utilized; 5) transport media is not required; 6) less than 1 colony forming unit is required for detection; 7) fast; and 8) economic.

This application claims priority to U.S. Provisional Patent Application Ser. No. 61/512,141, filed Jul. 27, 2011, which is incorporated by reference herein in its entirety.

### TECHNICAL FIELD

The field of the invention generally includes at least microbiology, cell biology, medicine, and diagnostics.

### BACKGROUND OF THE INVENTION

About 20 to 25% of women of childbearing age carry group B streptococcus (GBS) in their rectum or vagina during pregnancy and at delivery. Newborns acquire early-onset (EO) GBS infection in utero or during birth. About 50% of the newborns of maternal carriers are colonized on the skin or mucosal surface at birth if mother does not receive intrapartum antibiotic chemoprophylaxis (IAP). If mother receives IAP, this transmission rate decreases to about 5%. (CDC 1996, 2002, 2005, 2009, 2010) The transmission rate is greater for colonized women who have a vaginal versus Cesarean delivery if mother is not treated with IAP (45% vs. 10%) or treated with IAP (7% vs. 2%) (Lin 2011).

About 2% of colonized newborns develop early-onset GBS disease (onset within 7 days after birth); 98% of colonized newborns are said to be asymptomatic. The widespread use of intrapartum antibiotic prophylaxis since 1996 has reduced the incidence of EOGBS disease from about 1.8 per 1000 live births (LB) to 0.35 per 1000 LB. The incidence of late-onset disease (LOS) (7 to 180 days of age), however, remains the same or is slightly increased to about 0.3 per 1000 LB. (CDC 2005, 2009) Despite this recent decline in incidence of EOGBS disease, GBS remains a leading cause of bacterial sepsis and meningitis in newborns.

IAP has been the strategy of the U.S. National Guidelines for prevention of perinatal GBS disease (CDC 1996, 2002). The widespread use of IAP in the U.S has been accompanied by a reduction of neonatal EO GBS disease. The revised guidelines of 2002 and 2010 recommend universal screening of GBS at 35-37 weeks gestation and IAP to women who have had a positive prenatal GBS culture, had GBS bacteriuria during the current pregnancy, had an infant with invasive GBS disease previously, or whose GBS status is unknown and has any of the following clinical features: preterm delivery (<37 weeks gestation), ruptured membranes ˜18 hours or fever (>38.0° C.) during labor. TAP, however, is not recommended for prenatally GBS-positive women who undergo Cesarean delivery without labor or ruptured membranes (CDC 2002, 2010). These recommendations have been widely implemented in the U.S. A survey in 2003-2004 of selected counties in 10 states in the U.S. reported that 85.0% of women were screened for GBS before delivery and 85.1% of women who were eligible for antibiotic treatment during labor received chemoprophylaxis (Van Dyke 2009).

These guidelines, however, have limitations because prenatal cultures do not accurately predict GBS carriage during labor. Prior studies have reported that 4.0 to 11.6% of prenatally GBS-negative women had positive GBS cultures during labor (Allardice 1982, Boyer 1983, Yancey 1996, Goodman 1997, Edwards 2002, Hiller 2005, Towers 2010, Lin 2011), whereas 13.0 to 54.7% of prenatally GBS-positive women had a negative culture during labor (Table 1) (Allardice 1982, Boyer 1983, Yancey 1996, Goodman 1997, Edwards 2002, Hiller 2005, Towers 2010, Valkenburg 2006, E I Helali 2009, Lin 2011).

TABLE 1
Published positive and negative predictive values of prenatal culture for Group B streptococcal carriage at labor

| | | Gestational | Prenatal | Number | | Positive | Number | | Negative |
|---|---|---|---|---|---|---|---|---|---|
| | | age at | GBS + | positive cultures | | predictive | negative cultures | | predictive |
| Study | Location | culture | rate (%) | Prenatal | At labor | value (%) | Prenatal | at labor | value (%) |
| Allanlice | Canada | 28-34 | 10.3 | 53 | 29 | (45.3) | 471 | 452 | (96.0) |
| 1982 | | | | | | | | | |
| Boyer | U.S. | 1st-3rd | 22.8 | 393 | 264 | (67.2) | 200 | 183 | (91.9) |
| 1983 | | trimester | | | | | | | |
| Yancy | U.S. | 35-36 | 26.5 | 193 | 168 | (87.0) | 633 | 607 | (95.9) |
| 1996 | | | | | | | | | |
| Goodman | U.S. | 26-28 | 13.9 | 111 | 67 | (60.4) | 706 | 675 | (95.6) |
| 1997 | | | | | | | | | |
| Edwards | U.S. | 35-37 | NA | 218 | 146 | (67.0) | NA | NA | NA |
| 2002 | | | | | | | | | |
| Hiller | Australia | 36 | 20.0 | 120 | NA | (77.0) | 480 | NA | (94.0) |
| 2005 | | | | | | | | | |
| Valkenburg | Netherlands | 35-37 | 21.0 | 173 | 136 | (79.0) | 588 | 530 | (93.6) |
| 2006 | | | | | | | | | |
| El Helali | France | 35-37 | 12.3 | 115 | 67 | (58.3) | 818 | 753 | (92.1) |
| 2009 | | | | | | | | | |
| Towers | U.S. | Late 3^{rd} | 15.4 | 227 | 152 | (67.0) | 1245 | 1101 | (88.4) |
| 2010 | | trimester | | | | | | | |
| Lin | U.S. | ≧32 | 24.5 | 1172 | 592 | (50.5) | 3524 | 3233 | (91.7) |
| 2011 | | | | | | | | | |

Thus, some women who are colonized during labor will not receive IAP, subjecting their newborns to the risk of EOGBS disease. Those who are colonized prenatally but cultured negative during labor are given IAP despite no risk of delivering infants with EOGBS disease. Recent studies have reported that 61-82% of term newborns with EOGBS disease were born to prenatally GBS-negative mothers (Van Dyke 2009, Puopolo 2005, Pulver 2009). In a recent study routine prenatal GBS culture was compared with those cultures obtained during labor in a large population, but also assessed intrapartum antibiotic administration and mother-to-newborn transmission of GBS and evaluated these findings related to the prevention of EO GBS disease (Lin 2011). IAP was effective in interrupting mother-to-newborn transmission of GBS. However, ˜10% of prenatally GBS-negative women were positive during labor and missed IAP while −50% of prenatally GBS-positive women were negative during labor and received IAP unnecessarily. The inventors also observed that 93% of women who were GBS positive at 35-37 weeks gestation received IAP, while 20% of women who were GBS negative antepartum received antibiotics for reasons such as suspected maternal infection, Cesarean delivery, preterm labor or prolonged ruptured membranes. This resulted in about 38% of all pregnant women receiving antibiotics. These findings suggest that many woman receive intrapartum antibiotics, mostly for prevention of GBS disease, but the effectiveness of this strategy could be improved if a reliable screening system was available for rapid diagnosis of GBS colonization at the onset of labor.

Although the benefit of intrapartum antibiotic prophylaxis is impressive, a recent study conducted by NICHD scientists found that 8.8% of newborns colonized by GBS who did not develop EOGBS disease had signs of respiratory distress within 48 hours after birth and penicillin use during labor was associated with a 2.62 fold increase in respiratory distress in the colonized newborns. These findings suggested that GBS colonization and penicillin use during labor may have an adverse effect on newborns. (Lin 2006)

In support of these findings, experimental data showed that infusion of GBS into piglets and lambs resulted in pulmonary vasoconstriction and hypertension, decreased cardiac output and hypoxia. (Rojas 1984, Gibson 1989, Philips 1988, Rundle 1984, Tarpey 1987) Recently, Curtis and associates purified and identified phospholipids (cardiolipin and phosphatidylglycerol) from the GBS cell wall. Infusion of GBS phospholipids into baby lambs caused pulmonary hypertension. (Curtis 2003) Other scientists demonstrated that exposure of Streptococcus mutans to penicillin induces an immediate release of phospholipids from the bacteria (Cabacungan 1980, Horne 1977, Brissette 1982, Brissette 1985); cardiolipin and phosphatidylglycerol constitute more than one half of S. mutans phospholipids. (Brissette 1985) Although experimental data have shown the effect of penicillin on the release of phospholipids from S. mutans, extrapolating these data to clinical observation in human newborns required an assay that measures bacterial phospholipids in biologic specimens of GBS-colonized newborns.

Thus a rapid and reliable intrapartum GBS detection system in labor and delivery for mothers and babies would result in a significant improvement in the delivery of effective health care, by not treating culture-negative women, treating all culture-positive women, and identifying those infants who might be at risk for GBS culture negative respiratory distress.

Many tests have been evaluated including PCR, optical immunoassays (OIA), DNA probe, Latex Agglutination, enzyme immunoassays, ELISA, and Rapid Culture, but none have been developed to detect GBS rapidly from non-enriched samples except PCR. In fact the CDC 2010 guidelines for GBS screening suggests a supplemental role for such a nucleic acid amplification test (NAAT, aka PCR). Specifically it is stated that “In settings that can perform NAAT, such tests might prove useful for the limited circumstances of a woman at term with unknown colonization status and no other risk factors. The role proposed is limited because of the sensitivity of the assays in comparison to culture, but they also expressed concerns about real world turnaround time, test complexity, availability of testing at all times, staffing requirements, and costs.

At this time and for the foreseeable future, PCR appears to be the most logical and reliable method, and many have evaluated this technique for this indication. Table 2 summarizes these studies. (Bergeron 2000, Davies 2004, Convert 2005, Atkins 2006, Aziz 2005, Chan 2006, Gavino 2007, Edwards, 2008, Money 2008, Daniels 2009, El Helali 2009)

TABLE 2
Published positive and negative predictive values of Group B streptococcal culture versus PCR at labor

| | | Gestation | | Trans | Sample | | | % | No. + | No. + | | No. − | No. − | |
|---|---|---|---|---|---|---|---|---|---|---|---|---|---|---|
| Study | Location | (wks) | Swab | Media | Age | Rapid Test | n | Results | Cult. | RT | PPV | Cult. | RT | NPV |
| Our Data | Houston | ≧32 | Synthetic | None | Stored at | qPCR | 122 | 100 | 66 | 92 | 72 | 30 | 30 | 100 |
| Preliminary | | | | | 80° C. | | | | | | | | | |
| Daniels J | UK | >24 | triple | ? | Immed | Smart- | 1400 | 95.9 | 293 | 380 | 64.7 | 1049 | 562 | 95.1 |
| 2009 | | | | | | GBS ™ | | | | | | | | |
| El Helali | France | ≧35 | Copan | Amies | Immed | Xpert | 968 | 89.2 | 137 | 138 | 97.8 | 726 | 725 | 99.7 |
| 2009 | | | | | | GBS ™ | | | | | | | | |
| Money | Canada | ≧35 | | | | IDI | 190 | | | | | | | |
| 2008 | | | | | | Strep ™ | | | | | | | | |
| Edwards | USA | ≧35 | ? | ? | Immed | Xpert | 794 | 93 | 190 | 197 | 87.8 | 594 | 587 | 97.1 |
| 2008 | | | | | | GBS ™ | | | | | | | | |
| | | | | | | IDI | | | | | | | | |
| | | | | | | Strep ™ | | | | | | | | |
| Gavino | USA | ≧35 | Copan | None | Immed | Xpert | 55 | 100 ? | 24 | 34 | 67.6 | 31 | 21 | 95.2 |
| 2007 | | | | | | GBS ™ | | | | | | | | |
| Chan | UK | ≧37 | Probact | Probact | >24 hrs | PCR | 143 | 100 | 20 | 10 | 90 | 123 | 122 | 91.7 |
| 2006 | | | | | | | | | | | | | | |
| Atkins | USA | 9-43 | ? | ? | ≦24 hrs | IDI | 233 | 100 ? | 68 | 67 | 88.1 | 175 | 176 | 94.6 |
| 2006 | | | | | | Strep ™ | | | | | | | | |
| Aziz | USA | | | | | | 315 | | 56 | | | | | |
| 2005 | | | | | | | | | | | | | | |
| Convert | Switzerland | 33-37 | ? | Amies | Stored | PCR | 400 | 100 | 75 | 122 | 61.5* | 325 | 278 | 100 |
| 2005 | | | | | At 4° C. | | | | | | | | | |
| Davies | USA | >36 | Copan | Stuart | ≦24 hrs | IDI | 881 | 100 | 149 | 167 | 83.8 | 635 | 626 | 98.6 |
| 2004 | | | | | | Strep ™ | | | | | | | | |
| Bergeron | Canada | ? | Berton | Stuart | ≦24 hrs | PCR | 112 | 100 | 33 | 3? | 100 | 79 | 80 | 98.8 |
| 200 | | | | | | | | | | | | | | |

? = Unknown;
Immed = Immediately after collection

The issues around real world turn-around time, test complexity, availability of testing at all times, staffing requirements, and costs seem resolvable with an optimal PCR system if the issue of sensitivity can be addressed. Sensitivity seems to be influenced by several factors, including level of detection and inhibition. El Helali reported that 10.8% of samples resulted in no molecular diagnosis because of inhibition (40%), mucous (37%), and errors in loading (23%). Edwards reported interference from mucous, amniotic fluid, blood, lubricant ointments, and meconium. (News Release #2)

### BRIEF SUMMARY OF THE INVENTION

The present invention is directed to a system, method, and compositions for preparing a sample, including a biological sample. In particular embodiments, the sample is employed for testing for pathogens. In specific embodiments, the sample is employed for analysis of nucleic acid from the sample. Particular embodiments include preparation of samples from individuals that are suspected of having a pathogen or at risk for having the pathogen.

The invention is useful for detection of any pathogen, including for all bacteria (including mycoplasma), viruses and fungi, for example. The pathogen may be detected from a biological sample from an individual, including a mammal. The invention may be employed for a mammalian male or female, including human, cow, horse, dog, cat, sheep, goat, pig, and so forth. The invention may also be employed for a non-mammal, such as birds (chicken, turkey, etc.) and fish (salmon, tilapia, grouper, carp, catfish, seabass, and cod, for example).

In specific embodiments, the inventive process is an improvement over known methods because it provides accurate, rapid analysis that utilizes fewer steps and/or reagents from methods used in the art. In specific embodiments, it has one or more of the following characteristics: 1) it is a one-step process; 2) it eliminates dilution of the sample; 3) smaller sample sizes are employed; 4) fewer reagents are utilized; 5) transport media is not required; 6) less than 1 colony forming unit is required for detection; 7) fast; and 8) economic. In specific embodiments the sample is not diluted until the extraction process and only then minimally diluted in a small extraction buffer volume. In specific embodiments, the volume is between 20 and 200, 20 and 175, 20 and 150, 20 and 125, 20 and 100, 20 and 75, 20 and 50, 20 and 25, 30 and 200, 30 and 175, 30 and 150, 30 and 125, 30 and 100, 30 and 75, 30 and 50, 40 and 200, 40 and 175, 40 and 150, 40 and 125, 40 and 100, 40 and 75, 40 and 50, 50 and 200, 50 and 175, 50 and 150, 50 and 125, 50 and 100, 50 and 75, 60 and 200, 60 and 175, 60 and 150, 60 and 125, 60 and 100, 60 and 75, 75 and 200, 75 and 175, 75 and 150, 75 and 125, 75 and 100, 80 and 200, 80 and 175, 80 and 150, 80 and 125, 80 and 100, 100 and 200, 100 and 175, 100 and 150, 100 and 125, 125 and 200, 125 and 175, 125 and 150, 150 and 200, 150 and 175, or 175 and 200 μL, for example. In certain aspects, the volume is at least 5, 10, 20, 25, 30, 35, 40, 45, 50, 55, 60, 65, 70, 75, 80, 85, 90, 95, 100, 125, 150, 175, or 200 μL. In certain aspects, the volume is no more than 5, 10, 20, 25, 30, 35, 40, 45, 50, 55, 60, 65, 70, 75, 80, 85, 90, 95, 100, 125, 150, 175, or 200 μL.

In embodiments of the invention, the methods are employed for rapid diagnosis of infectious conditions. The source of these infections could be cultures taken from mucosal surfaces (e.g. vaginal, throat, conjunctiva, nasal, respiratory, tracheal, intestinal, stool, middle ear, etc.), wound surface cultures, urine cultures, sterile body fluid cultures (e.g. blood, cerebrospinal fluid, pleural fluid, peritoneal fluid, pericardial fluid, etc.). Therefore, samples from an individual in need of being tested for the presence of a pathogen may be obtained from these areas, including tissue, fluid, and so forth. The samples may come from the vagina, rectum, mouth, cervix, uterus, meconium, blood, urine, or skin, for example.

In certain embodiments, the present invention concerns methods and compositions for the improved identification of one or more pathogens in an expectant mother (for example, in the third trimester, including at approximately 35 to 37 weeks gestation or later), birthing mother, mother of a newborn infant, an in utero infant, or a newborn child. In specific embodiments, the invention concerns improved and rapid polymerase chain reaction analysis of samples from one or more individuals to detect a bacterial pathogen, including group B streptococcus. In particular aspects, the invention employs an optimized process for detection of group B streptococcus. In certain embodiments of the invention, the methods are a one-step extraction process for use in PCR for detection of group B streptococcus.

Contrary to conventional wisdom, in certain embodiments the present invention employs fewer rather than more steps in the preparation of the sample, whereas one would assume that the presence of contaminants would interfere with the PCR process. Also contrary to conventional thought, in particular embodiments the inventive process reduces the volumes utilized in various steps rather than using a larger volume to obtain more DNA. In addition, in certain embodiments, the inventive process utilizes fewer chemicals in the extraction solution.

In certain embodiments of the invention, the method is performed one or more times on an individual in need thereof. In specific cases, group B streptococcus can come and go, and the method of the invention is employed more than once. In certain cases, a pregnant mother has the test performed more than once, including in the third trimester and during delivery, for example.

In specific embodiments of the invention, there is a PCR non-enriched sample process that improves the level of detection and minimizes inhibition of the PCR. This process includes several steps including but not limited to: 1) use of a particular collection swab (Copan Swab; Murrieta, Calif.), 2) steps to reduce or minimize dilution of the swab-attached organisms (DNA), 3) a more effective DNA extraction solution, 4) elimination or significant reduction of interference from mucous, amniotic fluid, blood, lubricant ointments, and/or meconium, for example, and 5) a streamlined PCR process.

The inventors have demonstrated that the inventive PCR process can 1) detect<one cfu of organism (for example Group B streptococcus), and 2) is not interfered with by blood, albumin, amniotic fluid, mucous, etc. In addition, the inventors have tested this assay in 816 samples (205 positive by culture and 611 negative by culture) from a prior study that have been stored at −80° C. and observed a sensitivity of 100%; a specificity of 80%, a positive predictive value (PPV) of 63%, and a negative predictive value (NPV) of 100%; the sensitivity and NPV are the most important factors for a screening test.

This methodology is particularly useful for maternal GBS screening. It should be utilized for every woman who presents in labor as a more clinically comprehensive and cost-effective method for screening compared to current screening, including for those who need to receive IAP. It is also useful for every newborn as a means to screen for infants who remain at risk for GBS infection or in certain embodiments GBS cardiolipin-related respiratory distress. Thus, even with the development of alternate intervention strategies (e.g. a vaccine) in addition to or instead of IAP, this test has extensive application.

In some embodiments of the invention, there is a method of preparing a sample, comprising the steps of transporting a nondiluted sample from an individual to a sample analyzer, wherein the sample is transported on a swab having fibers with hydrophilic properties; placing the nondiluted sample directly in a nucleic acid extraction buffer; and extracting the nucleic acid in a single step. In specific embodiments, the extraction step comprises extraction with a buffer that comprises, consists essentially of, or consists of 10 mM Tris-HCL (pH 9.0), 50 mM KCl, 0.1% Triton® X-100, and 150 ng/μl Proteinase K In particular embodiments, the extraction buffer excludes one or more reagents commonly used in the art, such as ethylenediaminetetraacetic acid, sodium citrate, ethylene glycol tetraacetic acid, hydroxyethyl-ethylenediaminetriacetic acid, diethylene triamine pentaacetic acid, trisodium nitrilotriacetate, sodium lauryl sankosyl, sodium dodecyl sulfate, litium dodecyl sulfate, sodium glycocholate, sodium deoxycholate, sodium cholate, formamide, dimethyl sulfoxide, dithiothreitol, beta-mercaptoethanol, polyvinyl polypyrrolidone, ethanol, methanol, high pressure, high temperature, carbon dioxide, sodium citrate, lithium heparin, and/or sodium heparin. Thus this DNA extraction process eliminated unnecessary DNA purification steps and increased the sensitivity of the PCR.

In some embodiments of the invention, there is a method of preparing a sample, comprising the steps of obtaining a dry swab comprising a clinical sample from an individual suspected of being colonized by a microbe and preparing a diagnostic sample by extracting nucleic acid from the dry swab. In a specific embodiment, the dry swab comprises a rod and a plurality of hydrophilic fibers, wherein the fibers are substantially parallel to each other and normal to the surface of the rod. In certain cases, the nucleic acid is extracted using an extraction solution comprising: a. 10 mM Tris-HCL (pH 8.9, 9.0 or 9.1 or therebetween); b. 50 mM KCl c. 0.1% Triton® X-100; d. 150 ng/μl Proteinase K.

Sample preparation methods of the invention may further comprise analyzing the nucleic acid extracted from the sample, for example wherein analyzing the nucleic acid comprises polymerase chain reaction, sequencing, hybridization, microarray analysis, southern blot, northern blot, or a combination thereof.

Sample preparation methods of the invention may determine the presence or absence of one or more pathogens, in particular embodiments, and the pathogen may be selected from the group consisting of bacteria, virus, fungus, or a mixture thereof.

Samples prepared with methods of the invention may be obtained from the vagina, rectum, mouth, cervix, uterus, meconium, blood, urine, skin, amniotic fluid, joint fluid, ear canal, nasopharynx, cerebrospinal fluid, trachea, middle ear, occular fluid, anus, stool, intestine, stomach, or various tissues. In a specific embodiment, the sample is obtained from mucosal surfaces, placenta surfaces, wound surface cultures, urine cultures, sterile body fluid cultures. Exemplary mucosal surfaces are selected from the group of surfaces consisting of vaginal, throat, conjunctiva, nasal, respiratory, tracheal, intestinal, stool, and middle ear, in certain cases. Exemplary sterile body fluid cultures are selected from the group consisting of blood, cerebrospinal fluid, pleural fluid, peritoneal fluid, and pericardial fluid, in some cases.

In specific embodiments, the fiber of the dry swab comprises a synthetic polyamide polymer.

In specific embodiments of the invention, the individual is a pregnant mother, a mother of a newborn, or a newborn. The pregnant mother may be in the third trimester of gestation. The newborn may be suspected of having early or late onset group B streptococcus infection, in some cases.

In some embodiments, the volume of the extraction step is no more than 20-200 μL.

In particular embodiments of the invention, upon determination of the pathogen in the sample from the individual, the individual is treated for the presence of the pathogen.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The term “pathogen” as used herein refers to a disease-producing agent, including a bacterium, virus, fungus, or other microorganism.

The term “sample analyzer” as used herein refers to an individual or laboratory setting that analyzes a biological sample for the presence of a pathogen.

The term “swab” as used herein refers to material affixed to a stick for collection of specimen(s) from an individual. In specific embodiments, the material is a hydrophilic polymer.

Embodiments of the present invention include the preparation of a sample from an individual suspected of having a pathogen, including one at risk for developing deleterious symptoms from infection of the pathogen. The methods include transporting the sample without dilution to a laboratory setting, for example, such that the sample is then processed for extraction of nucleic acid prior to analysis of the nucleic acid. The extraction includes minimal volumes and there are no prior dilution or culture steps to remove contaminants and/or increase yield of the pathogen, yet the process is nevertheless effective, including for use of the nucleic acid in polymerase chain reaction, for example.

I. Group B Streptococcus (GBS)

Embodiments of the invention include assaying for bacteria, such as streptococcus. Streptococcus is a genus of spherical, Gram-positive bacteria of the phylum Firmicutes. Streptococcus agalactiae is a gram-positive streptococcus characterized by the presence of Group B Lancefield antigen. Group B Streptococcus (GBS), also known as Streptococcus agalactiae, Strep B, and group B Strep, can cause serious illness and sometimes death, particularly in newborn infants, the elderly, and patients with compromised immune systems (such as diabetes or cancer patients). Group B streptococci are also a hazard for veterinary pathogens, because they can cause bovine mastitis (inflammation of the udder) in dairy cows.

An infant born to a woman who is carrying the bacteria is at risk for contracting GBS. Some pregnant women have a higher risk of having a baby who develops group B strep disease, including if they have already had a baby with group B strep infection; have a urinary tract infection caused by group B strep; becomes colonized with group B strep late in pregnancy; develops a fever during labor; has rupture of membranes 18 hours or more before delivery; and/or begins labor or has rupture of membranes before 37 weeks.

II. Sample Extraction

In embodiments of the invention, a sample from an individual suspected of having a pathogen or at increased or general risk of having a pathogen is analyzed. The sample may be obtained from the source by the individual, laboratory, or institution performing the analysis or may be obtained elsewhere and transferred to the individual, laboratory, or institution performing the analysis.

The samples may be taken from any part of the individual so long as the sample harbors sufficient numbers of the pathogen to be detected by methods of the invention. The cultures may be taken from mucosal surfaces (e.g. vaginal, throat, conjunctiva, nasal, respiratory, tracheal, intestinal, stool, middle ear, ear canal etc.), wound surface cultures, urine cultures, placenta surfaces, sterile body fluid cultures (e.g. blood, cerebrospinal fluid, pleural fluid, peritoneal fluid, pericardial fluid, amniotic fluid, ophthalmic fluid, joint fluid, tissues (e.g. bone, brain, etc.), etc.). Therefore, samples from an individual in need of being tested for the presence of a pathogen may be obtained from these areas, including tissue, fluid, and so forth. In specific embodiments, the sample is obtained from the vagina, rectum, mouth, cervix, uterus, meconium, blood, cerebrospinal fluid, tracheal secretions, gastric aspirate, ear canal, nares, urine, or skin, for example.

The sample may be obtained from the individual by any means in the art, including by swab, needle, pick, scalpel, and so forth, but in specific embodiments the sample is obtained by a swab. In specific embodiments, a dry swab is utilized to obtain samples that are not diluted until the extraction process, such as samples from the vagina and/or rectum.

In particular embodiments, a swab is utilized in sample collection, such as one described in U.S. Patent Application Publication Number US 2006/0142668, which is incorporated by reference herein in its entirety. As described therein, the swab may be comprised of a solid molded plastic applicator shaft with a tip that can vary in size, shape, and layer of fiber, preferably of uniform thickness, and from 0.6 to 3 mm thick, for example. The fiber count, i.e. the weight in grams per 100 linear meters of a single fiber, is preferably between, 1.7 and 3.3 Dtex. In particular, a fiber of 0.6 mm length and 1.7 Dtex can be applied by flocking to obtain a fine nap, and a fiber up to 3 mm in length and 3.3 Dtex can be applied to obtain a long nap.

In specific cases of the swab, there is an ordered arrangement of the fibers, for example substantially parallel to each other and normal to the surface of the rod, avoiding any overlapping of fibers that can occur if the nap is too long. Indeed, in this manner the capillary represented by each fiber, by virtue of which it can carry out its task of absorbing and releasing essentially the same quantity of specimen, remains unimpaired and functional.

In particular embodiments, the fiber is chosen from a wide range of materials provided they are hydrophilic, such as, for example, synthetic or artificial materials, e.g. rayon, polyester, polyamide (including Nylon®), carbon fiber or alginate, natural materials e.g. cotton and silk, or mixtures thereof. In specific embodiments, the tip of the swab is coated with short Nylon® fibers that are arranged in a perpendicular fashion that results from a flocking process in which fibers are sprayed onto the tip of the swab while it is held in an electrostatic field. Such a process results in a highly absorbent thin layer having an open structure. In contrast to traditional fiber wound swabs, Copan Flocked Swabs have no internal absorbent core to disperse and entrap the specimen; the entire sample stays close to the surface for fast and complete elution. The perpendicular Nylon® fibers act like a soft brush and allow improved collection of samples. In embodiments wherein the sample comprises liquid, capillary action between the fiber strands facilitates strong hydraulic uptake of the liquid sample, and the sample stays close to the surface allowing easy elution.

III. Preparation of a Sample for Pathogen Analysis

In certain embodiments of the invention, there is a method that prepares a sample for analysis of one or more pathogens. In specific embodiments of the invention, there is a method that detects one or more pathogens using an improved process for preparing a sample for analysis of nucleic acid or protein from the sample, such as polymerase chain reaction analysis of nucleic acid, for example.

The method, at least in certain cases, utilizes a swab having hydrophilic fibers (such as a Copan Swab) for collection of the sample. The release of the preferably majority of the pathogens is allowed because of these swabs, and the sample may come from the vagina, rectum, or both, or amniotic fluid, in specific embodiments for Group B strep analysis. In newborns being tested, the sample may come from the throat, anus, stomach, nasopharynx, axilla, umbilicus, or external ear canal, for example before their first bath.

In particular cases of the invention, the sample is transported dry to the laboratory, yet preferably in a manner that excludes contamination from other sources. The swab having the sample may be encased in a tube, for example. In embodiments of the invention, the sample is not placed in any media between collection of the sample and the extraction process. At this point, the swab is placed directly into the extraction fluid, and in specific embodiments the extraction process is one step with reduced volume compared to methods in the art. In specific embodiments, the volume of extraction step is no more than between 20 and 200 μL.

In specific embodiments, the extraction fluid comprises, consists essentially of, or consists of 10 mM Tris-HCL (pH 9.0); 50 mM KCl; 0.1% Triton® X-100; 150 ng/μl Proteinase K; and water, such as distilled water. In specific embodiments, the concentrations are varied from these.

The extracted nucleic acid may then be employed for any process that utilizes detection of a pathogen, such as polymerase chain reaction, hybridization, sequencing, microarray analysis, southern blot, northern blot, and so forth.

Exemplary formulas to determine sensitivity and specificity are below:

| | | Condition | | |
|---|---|---|---|---|
| | | (as determined by “Gold standard”) | | |
| | | Positive | Negative | |
| Test out- come | Positive | True Positive | False Positive (Type I error) | $-> Positive predictive value = \frac{Σ True Positive}{Σ Test outcome Positive}$ |
| | Negative | False Negative (Type II error) | True Negative | $-> Negative predictive value = \frac{Σ True Negative}{Σ Test outcome Negative}$ |
| | | ↓ $Sensitivity = \frac{Σ True Positive}{Σ Condition Positive}$ | ↓ $Specificity = \frac{Σ True Negative}{Σ Condition Negative}$ | |

IV. Pathogens for Detection

The methods of the present invention utilize improved methods for sample analysis for the detection of one or more pathogens. In some cases, the pathogens include bacteria (including mycoplasma), viruses, fungi, a combination thereof, and so forth. The bacteria may be Gram-positive or Gram-negative bacteria. In specific embodiments, the pathogenic bacteria is one or more bacteria selected from the group consisting of the following phyla: 1) Aquificae; 2) Xenobacteria; 3) Fibrobacter; 4) Bacteroids; 5) Firmicutes; 6) Planctomycetes; 7) Chrysogenetic; 8) Cyanobacteria; 9) Thermomicrobia; 10) Chlorobia; 11) Proteobacteria; 12) Spirochaetes; 13) Flavobacteria; 14) Fusobacteria; and 15) Verrucomicrobia. In specific cases, the pathogen includes Gram positive cocci; Gram negative cocci; Gram positive bacilli; Gram negative bacilli, Spirochaetes, Rickettsia, or Mycoplasma. In certain embodiments, the present invention is useful against one or more bacteria that are resistant to one or more antibacterial agents, such as one or more antibiotics.

In certain cases, the pathogen is Staphylococcus, Streptococcus, Corynebacterium, Listeria, Bacillus, Clostridium, Neisseria, Enterobacteria, E. coli, Salmonella, Shigella, Campylobacter, Chlamydia, Borrelia, Francisella, Leptospira, Treponema, Proteus, Yersinia pestis, Vibrio, Helicobacter, Haemophila, Bordetella, Brucella, and Bacteriodes. In particular cases, the disinfectants are useful against Staphylococcus aureus, Listeria monocytogenes, Clostridium botulinum, Legionella pneumophila, E. coli, Salmonella enterica, Neisseria meningitides, Yersinia pestis, Mycobacterium tuberculosis, Vibrio cholera, Group A hemolytic streptococei, Diplococcus pneumonia, Moraxella catarrhalis, Neisseria gonorrhoeae, C. jeikeium, Mycobacterium avium complex, M. kansasii, M. leprae, M. tuberculosis, Nocardia sp, Acinetobacter calcoaceticus, Flavobacterium meningosepticum, Pseudomonas aeruginosa, P. alcaligenes, other Pseudomonas sp, Stenotrophomonas maltophilia, Brucella, Bordetella, Francisella, Legionella spp, Leptospira sp, Bacteroides fragilis, other Bacteroides sp, Fusobacterium sp, Prevotella sp, Veillonella sp, Peptococcus niger, Peptostreptococcus sp, Actinomyces, Bifidobacterium, Eubacterium, and Propionibacterium spp, Clostridium botulinum, C. perfringens, C. tetani, other Clostridium sp, Staphylococcus aureus (coagulase-positive), S. epidermidis (coagulase-negative), other coagulase-negative staphylococci, Enterococcus faecalis, E. faecium, Streptococcus agalactiae (group B streptococcus), S. bovis, S. pneumoniae, S. pyogenes (group A streptococcus), viridans group streptococci (S. mutans, S. mitis, S. salivarius, S. sanguis), S. anginosus group (S. anginosus, S. milleri, S. constellatus), Gemella morbillorum. Bacillus anthracis, Erysipelothrix rhusiopathiae, Gardnerella vaginalis (gram-variable), Enterobacteriaceae (Citrobacter sp, Enterobacter aerogenes, Escherichia coli, Klebsiella sp, Morganella morganii, Proteus sp, Providencia rettgeri, Salmonella typhi, other Salmonella sp, Serratia marcescens, Shigella sp, Yersinia enterocolitica, Y. pestis), Aeromonas hydrophila, Chromobacterium violaceum, Pasturella multocida, Plesiomonas shigelloides, Actinobacillus actinomycetemcomitans, Bartonella bacilliformis, B. henselae, B. quintana, Eikenella corrodens, Haemophilus influenzae, other Haemophilus sp, Mycoplasma pneumonia, Borrelia burgdorferi, Treponema pallidum Campylobacter jejuni, Helicobacter pylori, Vibrio cholerae, V. vulnificus, Chlamydia trachomatis, Chlamydophila pneumoniae, C. psittaci, Coxiella burnetii, Rickettsia prowazekii, R. rickettsii, R. typhi, R. tsutsugamushi, R. africae, R. akari, Ehrlichia chaffeensis, Ureaplasma urealyticum, Ureaplasma parvum, Ureaplasma diversum, Ureaplasma gallorale, U felinum, U cat, and U canigenitalium and Anaplasma phagocytophilum.

In particular embodiments of the present invention, the pathogen includes one or more pathogenic viruses. In specific embodiments, the one or more viruses is selected from the group consisting of Adenoviridae, Picornaviridae, Herpesviridae, Hepadnaviridae, Flaviviridae, Retroviridae, Orthomyxoviridae, Parvoviridae, Paramyxoviridae, Papovaviridae, Polyomavirus, Rhabdoviridae, and Togaviridae. Particular viruses include, for example, HIV, Adenovirus Influenza A, Rabies virus, Hepadnavirus, Varicella-zoster virus, Herpes simplex virus (types 1 and 2), Ebolavirus, Epstein Barr virus, Varicella-zoster virus, pox virus (including smallpox, copox, or monkey pox), human cytomegalovirus, poliovirus, coxsackievirus, Rubeola virus (paramyxovirus), Rubella virus, Variola virus, Avian flu virus (Influenza A virus), hepatitis A, B, and C viruses, parainfluenza, mumps virus, measles virus, respiratory syncitial virus, West Nile virus, Dengue fever virus, yellow fever virus, foot and mouth disease virus, human papiloma virus, and severe acute respiratory syndrome (SARS) coronavirus.

In particular embodiments of the present invention, the pathogen includes one or more pathogenic fungi. In specific embodiments, the antimicrobial agent is effective against one or more fungi selected from the group consisting of Histoplasma, Aspergillus and other common household molds, Candida, Cryptococcus, Stachybotrys, Zygomycosis, Fusarium, Blastomycosis, Coccidioides, Scedosporium, and Pneumocystis.

V. Therapy Following Detection

In some embodiments of the invention, the sample to be tested for the presence of one or more pathogens is prepared, and one or more pathogens is detected. The individual having a positive test for the pathogen may then be provided the appropriate therapy for the pathogen to prevent or reduce the severity of one or more symptoms of the infection. For pathogenic bacteria infections, one may receive one or more from one of the groups of aminoglycosides, carbapenems, cephalosporins, glycopeptides, lincosamides, macrolides, monobactams, nitrofurans, penicillins, quinolones, sulfonamides, tetracyclines, and so forth. Drugs against mycobacteria include, for example, clofazimine, isoniazid, rifampicin, streptomycin, and dapsone, for example.

Antiviral medications include Zanamivir, oseltamivir phosphate, Abacavir, Acyclovir, Adefovir, Amantadine, Amprevanir, Arbidol, Atazanir, Atripla, Bocoprevir, Cidofovir, Darunavir, Delavirdine, Didanosine, Edoxudine, Efavinerz, Emtricitabine, Enfuvirtide, Entecavir, Famciclovir, Fomivirsen, Fosmprenavir, Foscarnot, Ganciclovir, Immunovir, Idoxuridine, Indinavir, Maraviroc, Nelfinavir, Peginterferon alpha-2a, Pleconaril, Podophyllotoxin, Raltegravir, Ribavirin, Rimatadine, Ritonavir, Saquiravir, Stavudine, Tenofovir, Tipranavir, Trifluridine, Valaciclovir, Valganciclovir, Vidarabine, Viramidine, and Zidovudine.

In embodiments of the invention for Group B streptococcus, one may receive penicillin, cephalexin, or ampicillin, for example.

The therapies for pathogen treatment may be delivered by any means, but in specific embodiments they are provided intravenously, orally, intramuscular, intraocular, intravaginal, intraamniotic, intra joint, and so forth.

VI. Kits of the Invention

Any of the compositions described herein may be comprised in a kit. In a non-limiting example, a reagent and/or sample extraction tool may be comprised in a kit in suitable container means. In specific embodiments, the kit comprises a sample extraction tool, extraction buffer or reagent therefor, and/or polymerase chain reaction components. This includes any automated PCR detection system for pathogens.

The kits may comprise a suitably aliquoted extraction reagent composition of the present invention. The component(s) of the kits may be packaged either in aqueous media or in lyophilized form. However, the components of the kit may be provided as dried powder(s). When reagents and/or components are provided as a dry powder, the powder can be reconstituted by the addition of a suitable solvent. It is envisioned that the solvent may also be provided in another container means, in some embodiments.

The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the compositions and any other reagent containers in close confinement for commercial sale. Such containers may include injection or blow molded plastic containers into which the desired vials are retained.

In particular embodiments of the invention, a sample extraction tool is provided in the kit, including, for example, a swab, needle, pick, scalpel, and so forth. In specific embodiments, the swab comprises hydrophilic fibers attached to the shaft of the swab, including a Copan Swab, for example. In some embodiments, one or more components of an extraction fluid is provided including one or more of Tris-HCL (such as pH 9.0), Triton® X-100, KCl, proteinase, and water.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Methods and Reagents

In specific embodiments of the invention, there is a specialized sample process as follows:

1. A swab having hydrophilic (such as Nylon®) fibers (such as a Copan Swab; Murrieta, Calif.) is used to collect the samples. This allows all or almost all of the pathogen (at least for bacteria, 99+%) on the swab to be released. Normal cotton synthetic-tipped swabs release a small portion of the organisms.

2. The sample is transported dry to the laboratory, as opposed to placing the swab in transport media, for example a volume of more than one cc, such as 3 cc volume being standard in the art. Such a liquid transport from known methods dilutes out the organism concentration and thus negatively impacts its detectability (sensitivity).

3. The swab is not placed in any media initially for handling or growth prior to beginning the extraction process, and such a time period can be several minutes to hours. Again, this does not dilute the sample and does not delay the process.

4. The swab is placed directly into the extraction fluid, and in specific embodiments the extraction process is one step. Others in the art require several steps.

5. The volume of the extraction fluid is reduced compared to known methods and in specific embodiments can be approximately 50 μl. Others in the art employ a much larger volume, such as 10× to 20× as much as with the process of the invention, which again has the potential to result in sample dilution.

6. An exemplary embodiment of the extraction fluid contains the following items and concentrations:

a. 10 mM Tris-HCL (pH 9.0)

b. 50 mM KCl

c. 0.1% Triton® X-100

d. 150 ng/μl Proteinase K

e. distilled water as diluent

7. The extraction fluid (which contains the sample) is incubated at 55 to 58° C. for 15 to 20 minutes and then 95° C. for 5 minutes, in exemplary cases.

8. One can employ standard PCR probes and a standard target (for example, 200 bp). The cycle times and/or extension times may be optimized as is standard in the art.

The entire process can take less than 40 minutes but no more than 75 minutes, in specific embodiments. The inventors have used this process for several organisms. There is detection of less than or equal to 1 cfu per swab, and the negative predictive value and sensitivity are 100%, which is optimal for a screening test, such as screening for GBS in expectant mothers and/or newborns.

### Example 2

### Exemplary Ureaplasma Embodiments

The inventive methods were employed on the exemplary mycoplasma Ureaplasma. The inventors can detect less than 1 to 3 color changing units (ccu) with the inventive sample preparation method and subsequent PCR reaction.

From initial studies, the inventors tested 253 cultures for Ureaplasma with methods of the invention. Of those, 36 were positive (true positive) by culture and 54 were positive by PCR (including all of those that were positive by culture). Results were obtained for all samples, and there were no equivocal results.

Such results in the following:

Sensitivity: 100% (36)/(36+0)

Specificity: 92% (217)/(217+18)

Positive Predictive Value: 67% (36)/(36+18)

Negative Predictive value: 100% (217)/(217+0)

### Example 3

### Sensitive and Rapid Group B Streptococcus Intrapartum Detection System

Prenatal cultures may not accurately predict Group B Streptococcus (GBS) carriage during labor. It is known in the art that 4 to 11.6% of prenatal GBS-negative women are GBS culture positive during labor and do not receive intrapartum antibiotic prophylaxis (IAP) and also account for 61-82% of term newborns with early-onset GBS disease (EOGBS). It is also known that 13 to 54.7% of prenatal GBS-positive women are GBS culture negative during labor and may receive IAP unnecessarily.

A nucleic acid amplification test (NAAT) is useful at least for limited circumstances, particularly given the need for sensitivity; adequate turn around time; need for availability; and suitable cost.

The present invention provides an intrapartum GBS NAAT for non-enriched sample detection that is sensitive, rapid, and can be clinically available at low cost. The present invention provides an intrapartum GBS NAAT system for non-enriched sample detection that allows suitable sensitivity, specificity, negative predictive value, time to detect, and appropriate cost.

An exemplary clinical source of samples:

Vagino-rectal swab samples were collected on admission 24 hrs a day by healthcare providers from 2688 pregnant women>32 weeks gestation who: 1) presented for labor; 2) from Feb. 5, 2008 to Feb. 4, 2009; 3) at either Ben Taub General Hospital or St. Luke's Episcopal Hospital in Houston, Tex.; 4) maternal consent was obtained during prenatal visits or after admission for delivery. (Pediatr Infect Dis J 2011 30:759). The inventors randomly and blindly selected 816 vaginal samples from this study and compared the culture results with the present NAAT process.

Microbiological Procedures:

Standard microbiological techniques were used to identify GBS at a central microbiology laboratory. β-hemolytic colonies and suspicious nonhemolytic colonies were tested for GBS by latex agglutination (PathoDx, Diagnostics Product Corporation).Swabs were refrigerated and processed within 72 hrs. Each swab was placed in 2 ml of Todd-Hewitt Broth (THB) containing polymixin B (10 ug/ml), nalidixic acid (15 ug/ml), and crystal violet (0.1 ug/ml) and vortexed. 0.01 ml of broth was removed using a calibrated loop, streaked onto a colistin-nalidixic acid (CNA) agar plate, incubated at 37° C. for 24 hrs, and GBS colonies counted. If no GBS colonies were found on the CNA plate, 1 ml of the original THB, which had been incubated overnight, was subcultured onto a 5% sheep blood agar plate. Because GBS multiply in broth during incubation, subcultured plates were only interpreted as positive or negative.

Exemplary NAAT Process:

1 ml of the original THB was immediately frozen at −80° F. and used for this study. A process was developed via polymerase chain reaction (PCR) of non-enriched samples that included, for example:

1) use of an optimal commercially available collection swab

2) decreased dilution of the swab attached organisms/DNA

3) single step DNA extraction solution

4) streamlined PCR process

5) GBS Primers used for this study included:
a) Sag 59 and Sag 190 (D Ke, et al. Clin Chem, 2000)
b) DSF and DSR (F Kong, et al. J Clin Microbiol, 2002)

Statistics: Standard statistical analysis was used including Sensitivity, Specificity, Negative Predictive Value, Positive Predictive Value.

Exemplary Results

Bench-top preclinical studies of PCR process resulted in detection of <1 cfu per swab of GBS and no detectable interference from albumin; amniotic fluid; blood; lubricants; meconium; and mucous.

Culture and qPCR of 816 vaginal-rectal clinical samples:

| | | | qPCR | | qPCR |
|---|---|---|---|---|---|
| | N | | Positive | | Negative |
| Culture Results | (% of all samples) | | (% of all samples) | | (% of all samples) |
| Positive | | | | | |
| Broth Only | 86 | (11) | 86 | (11) | 0 |
| 1-50 CFR | 76 | (9) | 76 | (9) | 0 |
| 51-100 CFR | 22 | (3) | 22 | (3) | 0 |
| >100 CFR | 17 | (2) | 17 | (2) | 0 |
| Subtotal | 205 | (25) | 205 | (25) | 0 |
| Negative | 611 | (75) | 123 | (15) | 488 (60) |
| Total samples | 816 | (100) | 328 | (40) | 488 (60) |

In the detailed analysis for the randomly selected 816 samples where culture on admission was negative but GBS PCR was positive (false positive) n=123: prenatal GBS culture was positive in 30 of 98 (31%), and prenatal GBS culture was negative in 68 of 98 (25 of these 68 received intrapartum antibiotics (37%)). No prenatal GBS culture were available in 25.

PCR evaluation of clinical samples found a result for each sample tested; generally <40 but up to 75 min to complete each sample in laboratory; PCR positive for all 205 culture positive samples; sensitivity is 1.0 (95% CI: 1.0-0.98); negative predictive value of 1.0 (95% CI: 1.0-0.99); specificity: 0.80 (95% CI: 0.76-0.83); and a cost estimation that for hospital with 4000 births annually, the cost of each sample would be $65 (including supplies, staff, equipment investment).

Therefore, a sensitive, rapid, low-cost intrapartum GBS NAAT process was developed and tested in 816 non-enriched vaginal samples. 100% sensitivity and negative predictive value makes this an ideal screening test.

In some embodiments, the observed 80% specificity may be due to persistent GBS antigen in previously colonized GBS; antepartum antibiotics suppressing GBS growth; and/or some positive GBS cultures that failed to grow. In embodiments of the invention, this GBS NAAT process decreases EOGBS disease and/or unnecessary IAP.

In some embodiments, one may be able to improve the PCR efficiency and specificity of the claimed invention: 1) optimize concentration of primers and reagents in current PCR process to improve specificity and efficiency, by varying concentrations of these components for multiple samples; 2) investigate other commercially available primers and reagents to determine if one can improve specificity and efficiency; 3) modify primers (e.g.): a) adding AT rich flaps on the 5′ end of primers to significantly improve SYBER green qPCR results [Alfonina, 2007]; b) linkers attached within primers to create tethered segments can increase specificity [Chun 2007]; c) tripeptide modification or minor groove binding modification can increase specificity [Kutayvin 2000]; d) locked nucleic acid modifications show greatly enhanced thermal stability and can increase specificity [Kaur 2006]; and/or e) ensure avoidance of unwanted primer homologies to improve specificity [Bikandi 2004]; 4) improve reagents (e.g.): a) using reagents like anti-Taq antibodies or heat inactivated polymerases or dNTPs can increase specificity; b) optimizing concentration of polymerase, magnesium and dNTPs could increase specificity [Kunz 1991, Markoulatos 2002]; c) use adjuvants such as betadine, dimethyl sulfoxide, etc to increase PCR efficiency. [Demke 1992, Henke 1997]; 5) optimize the thermal cycling conditions by performing annealing temperature gradients. This could result in improved specificity and efficiency. [Wittwer 1991]; 6) ensure primers have similar stabilities (Tₘ within 1 to 2 degrees) to improve specificity [Hecker 1996]; 7) evaluate the final process results across several common commercially available PCR instruments. There may be additional steps that develop over time that could improve the efficiency and specificity of this process and they can be evaluated and included as appropriate. This will ensure the sustainability of embodiments of a process for clinical use by the multiple instruments utilized by clinical laboratories, for example.

### REFERENCES

The following references, to the extent that they provide exemplary procedural or other details supplementary to those set forth herein, are specifically incorporated herein by reference.

### PATENT APPLICATIONS

US 2006/0142668

### PUBLICATIONS

Allardice J G, Baskett T F, Seshia M M, Bowman N, Malazdrewicz R. Perinatal group B streptococcal colonization and infection. Am J Obstet. Gynecol. 1982; 142(6 Pt 1):617-20.
Atkins K L, Atkinson R M, Shanks A, Parvin C A, Dunne W M, Gross G. Evaluation of polymerase chain reaction for group B streptococcus detection using an improved culture method. Obstet. Gynecol. 2006; 108:488-91.
Aziz N, Baron E J, D'Souza H, Nourbakhsh M, Druzin M L, Benitz W E. Comparison of rapid intrapartum screening methods for group B streptococcal vaginal colonization. J Matern Fetal Neonatal Med. 2005; 18(4):225-9.
Bergeron M G, Ke D, Menard C, Picard F J, Gagnon M, Bernier M, Ouellette M, Roy P H, Marcoux S, Fraser W D. Rapid detection of group B streptococci in pregnant women at delivery. N Engl J. Med. 2000 JuI20; 343(3):175-9.
Boyer K M, Gadzala C A, Kelly P D, Burd 1I, Gotoff S P. Selective intrapartum chemoprophylaxis of neonatal group B streptococcal early-onset disease. II. Predictive value of prenatal cultures. J Infect Dis. 1983; 148(5):802-9.
Brissette J L, Shockman G D, Pieringer R A. Effects of penicillin on synthesis and excretion of lipid and Iipoteichoic acid from Streptococcus mutans B HT. J. Bacteriol. 1982; 151:838-844.
Brissette J L, Pieringer R A. The effect of penicillin on fatty acid synthesis and excretion in Streptococcus mutans. Lipids. 1985; 20:173-179.
Cabacungan E, Pieringer R A. Excretion of extracellular lipids by Streptococcus mutans B HT and F A-1. Infect Immun. 1980; 27:556-562.
Centers for Disease Control and Prevention. Prevention of perinatal group B streptococcal disease. MMWR Morb Mortal Wkly Rep 1996; 45: (RR-7): 1-24.
Centers for Disease Control and Prevention. Prevention of perinatal group b streptococcal disease. Revised guidelines from CDC. MMWR Morb Mortal Wkly Rep 2002; 51(RR-11):1-24.
Centers for Disease Control and Prevention. Rate of early-onset and late-onset invasive group B streptococcal disease in infants, by year, 1996-2004, Active Bacterial Core Surveillance system, United States. MMWR Morb Mortal Wkly Rep 2005; 54(47):1205-08.
Centers for Disease Control and Prevention. Trends in perinatal group B streptococcal disease—United States, 2000-2006. MMWR Morb Mortal Wkly Rep 2009; 58(05):109-112.
Centers for Disease Control and Prevention. Prevention of perinatal group B streptococcal disease. MMWR Morb Mortal Wkly Rep 2010; 59: (RR-10): 1-32.
Centers for Disease Control and Prevention. Prevention of Perinatal Group B Streptococcal Disease. MMWR Morb Mortal Wkly Rep 2011 59(RR-10):1-32.
Chan K L, Levi K, Towner K J, Weston V C, Ramsay M M, Kean L H. Evaluation of the sensitivity of a rapid polymerase chain reaction for detection of group B streptococcus. J Obstet. Gynaecol. 2006; 26(5):402-6.
Convert M, Martinetti Lucchini G, Dolina M, Piffaretti J C. Comparison of LightCycler PCR and culture for detection of group B streptococci from vaginal swabs. Clin MicrobiolInfect. 2005 December; 11 (12): 1022-6.
Curtis J, Kim G, Wehr N B, Levine R L. Group B streptococcal phospholipid causes pulmonary hypertension. PNAS. 2003; 100:5087-5090.
Daniels J, Gray J, Pattison H, Roberts T, Edwards E, Milner P, Spicer L, King E, Hills R K, Gray R, Buckley L, Magill L, Elliman N, Kaambwa B, Bryan S, Howard R, Thompson P, Khan K S.
Rapid testing for group B streptococcus during labour: a test accuracy study with evaluation of acceptability and cost-effectiveness. Health Technol Assess. 2009 September; 13(42):1-154, iii-iv
Davies H D, Miller M A, Faro S, Gregson D, Kehl S C, Jordan JAMulticenter study of a rapid molecular-based assay for the diagnosis of group B Streptococcus colonization in pregnant women. Clin Infect Dis. 2004 Oct. 15; 39(8): 1129-35.
Edwards R K, Clark P, Duff P. Intrapartum antibiotic prophylaxis 2: positive predictive value of antenatal group B streptococci cultures and antibiotic susceptibility of clinical isolates. Obstet. Gynecol. 2002; 100(3):540-4.
E I Helali N, Nguyen J C, Ly A, Giovangrandi Y, and Trinquart L. Diagnostic accuracy of a rapid real-time polymerase chain reaction assay for universal intrapartum group B streptococcus screening. Clin Infect Dis 2009; 49:417-23.
Gavino M, Wang E. A comparison of a new rapid real-time polymerase chain reaction system to traditional culture in determining group B streptococcus colonizationAm J Obstet. Gynecol. 2007 October; 197(4):388.e1-4.
Gibson R L, Truog W E, Redding G J. Thromboxane-associated pulmonary hypertension during three types of Gram-positive bacteremia in piglets. Pediatr Res. 1989; 23:553-556.
Goodman J R, Berg R L, Gribble R K, Meier P R, Fee S C, Mitchell P D. Longitudinal study of group B streptococcus carriage in pregnancy. Infect Dis Obstet. Gynecol. 1997; 5(3):237-43.
Hiller J E, McDonald H M, Darbyshire P, Crowther CAAntenatal screening for Group B Streptococcus: a diagnostic cohort study. BMC Pregnancy Childbirth. 2005; 5:12.
Horne D, Hakenbeck R, Tomasz A Secretion of lipids induced by inhibition of peptidoglycan synthesis in streptococci. J. Bacteriol. 1977; 132:704-717.
Lin F Y, Weisman L E, Azimi P, Young A E, Chang K, Cielo M, Moyer P, Troendle J F, Schneerson, Robbins J B. Assessment of Intrapartum Antibiotic Prophylaxis for the Prevention of Early-onset Group B Streptococcal Disease. Pediatr Infect Dis J 2011 30:1-5.
Lin F Y & Troendle J F. Hypothesis: Neonatal respiratory distress may be related to asymptomatic colonization with group B streptococci. Pediatr Infect dis J 2006; 25:884-888.
Money 0, Dobson S, Cole L, et al. An evaluation of a rapid real time polymerase chain reaction assay for detection of group B streptococcus as part of a neonatal group B streptococcus prevention strategy. J Obstet Gynaecol Can. 2008; 30(9):770-5.
Philips J B III, Lyrene R K, Godoy G, et al. Hemodynamic responses of chronically instrumented piglets to bolus injections of group B streptococci. Pediatr Res. 1988; 23:81-85.
Pulver L S, Hopfenbeck M M, Young P C, Stoddard G J, Korgenski K, Daly J, Byington C L. Continued early onset group B streptococcal infections in the era of intrapartum prophylaxis. J. Perinatol. 2009; 29(1):20-5.
Puopolo K M, Madoff L C, Eichenwald E C. Early-onset group B streptococcal disease in the era of maternal screening. Pediatrics. 2005; 115(5):1240-6.
Rundle B, Goldbery R N, Streitfeld M M, et al. Cardiovascular changes in group B streptococcal sepsis in the piglet: Response to indomethacin and relationship to prostacyclin and thromboxane A2. Pediatr Res. 1984; 18: 874-878.
Tarpey M N, Graybar G B, Lyrene R K, et al. Thromboxane synthesis inhibition reverses group B streptococcus-induced pulmonary hypertension. Crit Care Med. 1987; 15:644-647.
Towers C V, Rumney R N C, Asrat T. Preslicka C, Ghamsary M G, and Nageotte M P. The accuracy of late third-trimester antenatal screening for group B streptococcus in predicting colonization at delivery. Am J Perinatol. 2010; 27(10):789-90.
Van Dyke M K, Phares C R, Lynfield R, et al. Evaluation of universal antenatal screening for group B streptococcus. N Engl J Med. 2009; 360(25):2626-36.
Valkenburg-van den Berg A W, Sprij A J, Oostvogel P M, et al. Prevalence of colonisation with group B Streptococci in pregnant women of a multi-ethnic population in The Netherlands. Eur J Obstet Gynecol Reprod Biol. 2006; 124(2):178-83.
Yancey M K, Schuchat A, Brown L K, Ventura V L, Markenson G R. The accuracy of late antenatal screening cultures in predicting genital group B streptococcal colonization at delivery. Obstet Gynecol. 1996; 88(5):811-5.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of preparing a sample, comprising the steps of:
obtaining a dry swab comprising a clinical sample from an individual suspected of being colonized by a microbe;
preparing a diagnostic sample by extracting nucleic acid from the dry swab.

2. The method of claim 1, wherein the dry swab comprises a rod and a plurality of hydrophilic fibers, wherein the fibers are substantially parallel to each other and normal to the surface of the rod.

3. The method of claim 1, wherein the nucleic acid is extracted using an extraction solution comprising:
a. 10 mM Tris-HCL (pH 8.9 or 9.1)
b. 50 mM KCl
c. 0.1% Triton® X-100
d. 150 ng/μl Proteinase K

4. The method of claim 1, further comprising analyzing the nucleic acid extracted from the sample.

5. The method of claim 4, wherein analyzing the nucleic acid comprises polymerase chain reaction, sequencing, hybridization, microarray analysis, southern blot, northern blot, or a combination thereof.

6. The method of claim 5, wherein analyzing the nucleic acid comprises conducting polymerase chain reaction.

7. The method of claim 4, wherein the presence or absence of one or more pathogens is determined.

8. The method of claim 7, wherein the pathogen is selected from the group consisting of bacteria, virus, fungus, or a mixture thereof.

9. The method of claim 8, wherein the sample is obtained from the vagina, rectum, mouth, cervix, uterus, meconium, blood, urine, skin, amniotic fluid, joint fluid, ear canal, nasopharynx, cerebrospinal fluid, trachea, middle ear, occular fluid, anus, stool, intestine, stomach, or various tissues.

10. The method of claim 1, wherein the sample is obtained from mucosal surfaces, placenta surfaces, wound surface cultures, urine cultures, sterile body fluid cultures.

11. The method of claim 10, wherein the mucosal surfaces are selected from the group of surfaces consisting of vaginal, throat, conjunctiva, nasal, respiratory, tracheal, intestinal, stool, and middle ear.

12. The method of claim 10, wherein the sterile body fluid cultures is selected from the group consisting of blood, cerebrospinal fluid, pleural fluid, peritoneal fluid, and pericardial fluid.

13. The method of claim 2, wherein the fiber comprises a synthetic polyamide polymer.

14. The method of claim 1, wherein the individual is a pregnant mother, a mother of a newborn, or a newborn.

15. The method of claim 14, wherein the pregnant mother is in the third trimester of gestation.

16. The method of claim 14, wherein the newborn is suspected of having early onset group B streptococcus infection.

17. The method of claim 14, wherein the newborn is suspected of having late onset group B streptococcus infection.

18. The method of claim 7, wherein the pathogen is group B streptococcus.

19. The method of claim 2, wherein the volume of the extraction step is no more than 20-200 μL.

20. The method of claim 7, wherein upon determination of the pathogen in the sample from the individual, the individual is treated for the presence of the pathogen.
